(19) United States
(12) Patent Application Publication
(10) Pub. No.: US 2008/0118590 A1
(43) Pub. Date: May 22, 2008

(54) Using composition containing cAMP (cyclic adenosine monophosphate), nicotinic acid, prostaglandin, gamma linolenic acid and xanthines in weight loss and/or reducing elevated triglyceride

(76) Inventor: Chaosong Hsue, Taichung (TW)

Correspondence Address: BACON & THOMAS, PLLC, 625 SLATERS LANE, FOURTH FLOOR, ALEXANDRIA, VA, 22314, US

(21) Appl. No.: 11/984,866

(22) Filed: Nov. 23, 2007

(30) Foreign Application Priority Data
Nov. 22, 2006 (TW) 095143211

(51) Int. Cl. A61K 36/725; A61K 36/28; A61K 125/00; A61P 3/00; A61K 36/232

(52) U.S. Cl. 424/773; 424/725

(57) ABSTRACT

An using composition containing cAMP (Cyclic Adenosine Monophosphate), Nicotinic Acid, Prostaglandin, Gamma Linolenic Acid as effective ingredient in management of weight reduction or hypertriglyceridemia, furthermore disclose of certain natural products are essentially contain of said ingredients are to be used for such purpose.

For incidence, coffee is the source of caffeine, tea is the source of theophylline, and cocoa is the source of theobromine. Zizyphus jujube mill contains rich amount of cAMP, Radix Angelica Sinensis, Sunflower, Matrimony vine (Lycium chinense Mill), Edible fig (Ficus carica) are sources of Nicotinic Acid, Evening primrose, Starflower contain Gamma Linolenic Acid which are indirect sources of Prostaglandin.

This invention discloses the random combination use of the above composition is useful and effective in the management of weight reduction or reducing elevated triglyceride.

### FIELD OF THE PRESENT INVENTION

The present invention is using composition containing cAMP (Cyclic Adenosine Monophosphate), Nicotinic Acid, Prostaglandin, Gamma Linolenic Acid as effective ingredient in management of weight reduction or hypertriglyceridemia.

### BACKGROUND OF THE PRESENT INVENTION

1. PPA (Phenylpropanolamine), related to a sympathetic nerve stimulant essentially by effecting the hypothalamus nerve to create lack of appetite and frustrated the desire for food so as to cut down the amount of food taking, thus to achieve the purpose of weight reduction.

2. Xanical (Orlistat), related to an inhibitor for the lipase from pancreas in the digestion duct by preventing fat in food from being decomposed and absorbed by the human body, furthermore, the oily excrement is common.

3. Reductil (Subutramine), related to an inhibitor to the absorption of serum serotonin and nor adrenaline, both conduction substances of the nerves system, functioning by developing the sense of satiety and thermo genesis.

4. Adenosine 5′-triphosphate (ATP), related to decrease the sensitivity (desensitization) of adenosine with A1 adenosine receptors of fat cells.

5. cAMP (Cyclic Adenosine triphosphate), related to activation of triglyceride lipase.

6. Xanthines, relate to inhibition of phosphodiesterase.

### SUMMARY OF THE INVENTION

The primary purpose of the present invention is to use natural products in the management of weight reduction or reducing elevated triglyceride level in human body. cAMP activates triglyceride lipase, encourages the metabolism of lipid and decomposition of triglyceride into FFA and glycerol which decrease the volume of fat cells.

Xanthines inhibit phosphodiesterase which increase the concentration of cAMP.

Nicotinic Acid, prostaglandin induces the synthesis of cAMP promotes lipid metabolism as well.

Gamma Linolenic Acid stimulates the synthesis of prostaglandin, increases the level of cAMP also facilitates the metabolism of triglyceride in human body.

The present invention demonstrates the biochemical pathway of certain natural products are new and useful in the management of weight reduction or reducing the elevated level of triglyceride.

### BRIEF DESCRIPTION OF THE DRAWINGS

FIG. 1. Is the relationship of triglyceride metabolism pathway with cAMP, nicotinic acid, prostaglandin and xanthines.

FIG. 2. Is the molecular structure of cAMP (Cyclic Adenosine Monophosphate)

FIG. 3. Is the molecular structure of Nicotinic Acid (Nicotinamide, Vitamin B3)

FIG. 4. Is the molecular structure of Prostaglandin (PGE1)

FIG. 5. Is the molecular structure of Caffeine (1,3,7-trimethyl-xanthine)

FIG. 6. Is the molecular structure of Theophylline (1,3-dimethyl-xanthine)

FIG. 7. Is the molecular structure of Theobromine (3,7-dimethyl-xanthine)

### DESCRIPTION OF THE PREFERRED EMBODIMENT

The present invention comprising cAMP (Cyclic Adenosine Monophosphate), Niacin (Nicotinic Acid, Nicotinamide), Prostaglandin, and Xanthines whereas:

Xanthines are Coffee, Tea and Cocoa.

cAMP is Zizyphus.

Niacin are radix Angelica, Sunflower, Matrimony vine (lycium chinense) and Edible Fig (Ficus Carica).

The synthesized of prostaglandin can be stimulated by Gamma Linolenic Acid.

Gamma Linolenic Acid are Evening primrose and Starflower.

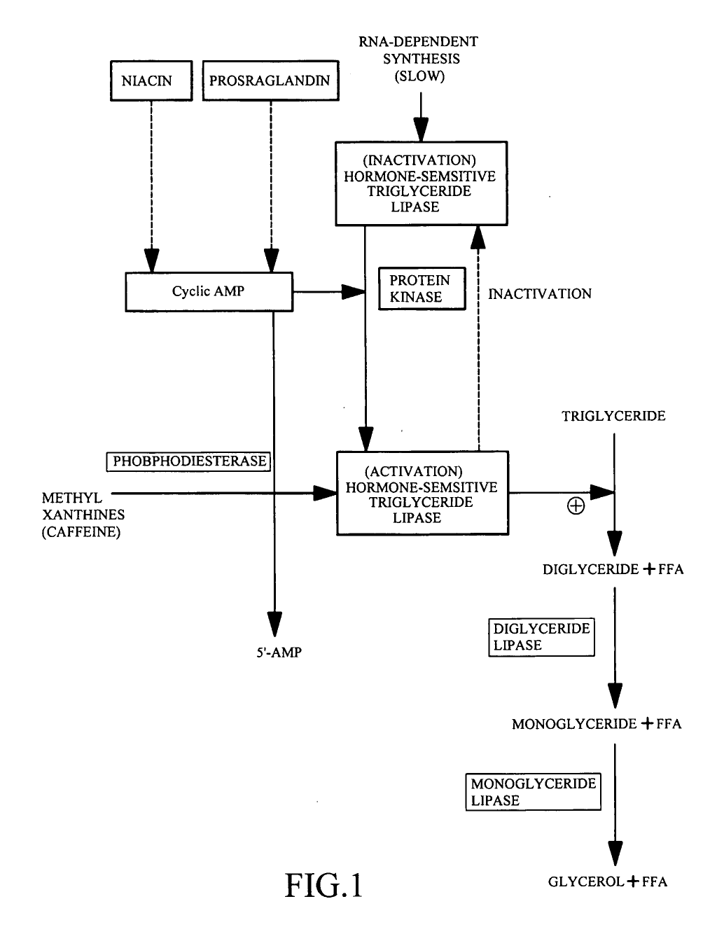

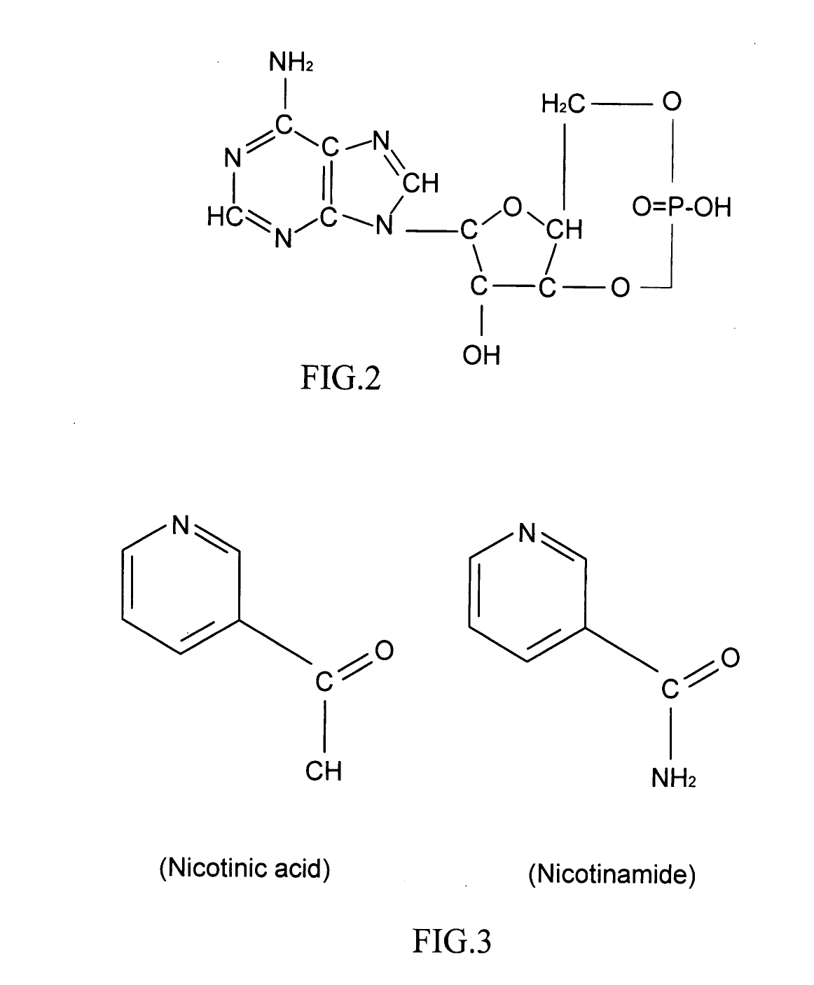

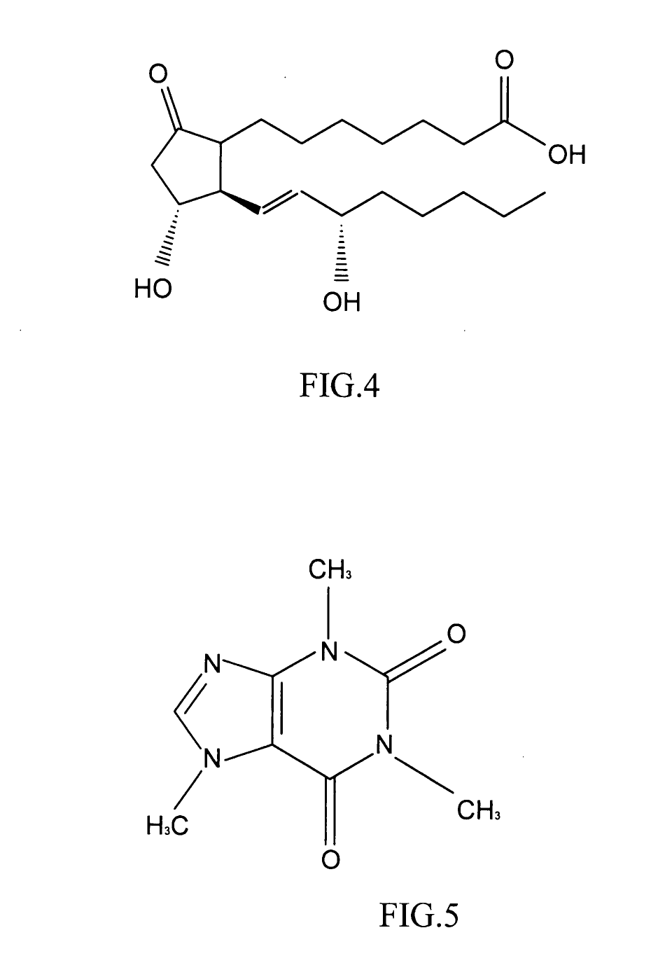

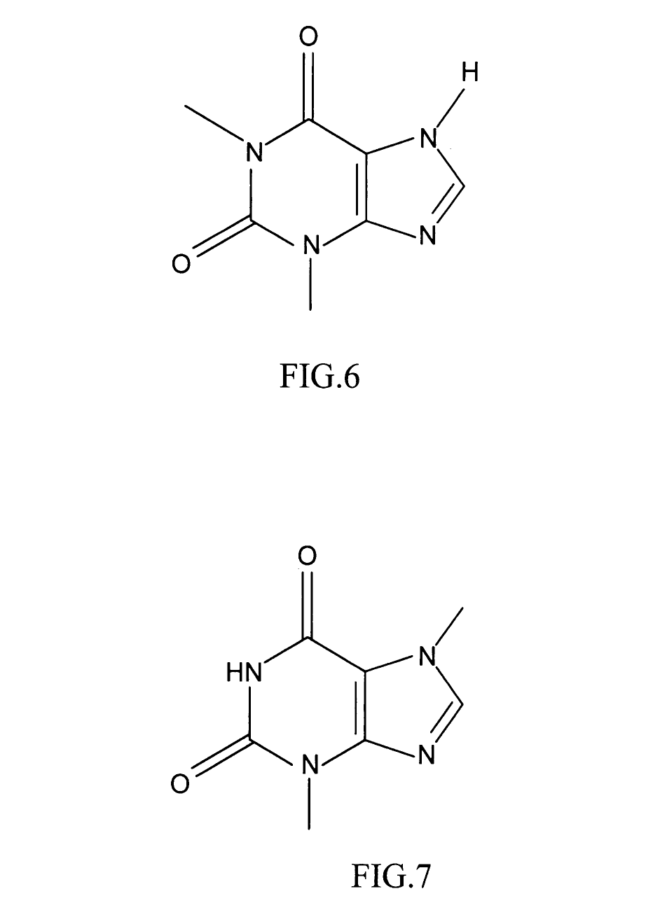

When administration of Zizyphus results the elevation of body concentration of cAMP, the cAMP in human body activates certain protein kainase and triglyceride lipase, then triglyceride decomposed into diglyceride, monoglyceride, then glycerol and FFA. The triglyceride in adipose tissue metabolized, the volume of fat cell decreased. The triglyceride metabolism could be enhanced with the presence of xanthines (coffee, tea, cocoa), because xanthine inhibit phosphodiesterase, inhibit the formation of 5′ AMP which has no effect on activation of triglyceride lipase as illustrated in FIG. 1.

When administration of radix Angelica, Sunflower, Matrimony vine (lycium chinense) or Edible Fig (Ficus Carica) with coffee, tea or cocoa, increases the body concentration of cAMP and xanthines. The presence of xanthine inhibits phosphodiesterase and elevates the concentration of cAMP which activates triglyceride lipase and triglyceride metabolism then decrease size and volume of fat cells, reducing the level of triglyceride in the blood circulation.

When administration of evening primrose or starflower with coffee, tea or cocoa, increases the body concentration of prostaglandin, cAMP and xanthines. The presence of xanthine inhibits phosphodiesterase and elevates the concentration of cAMP which activates triglyceride lipase and triglyceride metabolism then decrease size and volume of fat cells, reducing the level of triglyceride in blood circulation.

The present invention demonstrates the metabolism of triglyceride with indigestion of certain natural products is new and useful in the management of over weight and reducing elevated triglyceride level in human body is different from related prior art.

The evidence based knowledge taught us that the natural products of this invention has been used safely over thousands of years and is acceptable on dinning table worldwide.

1. A composition used in management of weight reduction or reducing elevated triglyceride containing Coffee and Zizyphus and or its extract.

2. A composition used in management of weight reduction or reducing elevated triglyceride containing Coffee and Radix Angelica and or its extract.

3. A composition used in management of weight reduction or reducing elevated triglyceride containing Coffee and Sunflower and or its extract.

4-21. (canceled)
